(19) United States
(12) Patent Application Publication
(10) Pub. No.: US 2012/0034642 A1
(43) Pub. Date: Feb. 9, 2012

(54) METHOD FOR OBTAINING A SINGULAR CELL MODEL CAPABLE OF REPRODUCING IN VITRO THE METABOLIC IDIOSYNCRASY OF HUMANS

(75) Inventors: JOSÉ VICENTE CASTELL RIPOLL, VALENCIA (ES); MARÍA JOSÉ GÓMEZ-LECHÓN, VALENCIA (ES); RAMIRO JOVER ATIENZA, VALENCIA (ES); AGUSTÍN LAHOZ RODRÍGUEZ, VALENCIA (ES)

(73) Assignee: ADVANCED IN VITRO CELL TECHNOLOGIES, S.L., Barcelona (ES)

(21) Appl. No.: 13/175,138

(22) Filed: Jul. 1, 2011

Related U.S. Application Data
(62) Division of application No. 10/597,286, filed on Oct. 20, 2006, now abandoned.

(30) Foreign Application Priority Data
Jan. 19, 2004 (EP) PCT/EP2004/000339

(51) Int. Cl. C12N 5/10; C12Q 1/02; C12N 15/861; C12N 15/85

(52) U.S. Cl. 435/29; 435/455; 435/370; 435/456; 435/320.1

(57) ABSTRACT

The method is based on the use of expression vectors coding for the sense and anti-sense mRNA of the Phase I and Phase II drug biotransformation enzymes showing a greatest variability in humans for transforming cells expressing reductase activity. Such vectors can modulate (increase or decrease) the individualized expression of an enzyme without affecting the other enzymes. This singular cell model can reproduce in vitro the metabolic idiosyncrasy of humans. It is applicable in the study of development of new drugs, specifically in the study of metabolism, potential idiosyncratic hepatotoxicity, medicament interactions, etc., of new drugs.

### CROSS-REFERENCE TO RELATED APPLICATIONS

This application is filed under the provisions of 35 U.S.C. §120 and is a divisional of U.S. patent application Ser. No. 10/597,286 filed Jul. 19, 2006 in the name of José Vicente CASTELL RIPOLL et al., which was filed under the provisions of 35 U.S.C. §371 and claims the priority of International Patent Application No. PCT/EP2004/000339 on Jan. 19, 2004, which are both hereby incorporated by reference herein in their entireties.

### FIELD OF THE INVENTION

The invention relates to obtaining a singular cell model capable of reproducing in vitro the metabolic idiosyncrasy of humans by expression vectors that encode for the sense and anti-sense mRNA of the enzymes of the drug biotransformation Phases 1 and II showing greatest variability in humans. This approach, based in the use of viral expression vectors, allows also to confer to any cell type (tumoral or not), of any tisular origin, the ability to express Phase 1 and/or Phase II biotransformation enzymes with activity against xenobiotics. When the mentioned biotransformation enzymes are CYP enzymes, it is necessary that, in addition, cells to be transfected show or express enough cytochrome P450 reductase activity. In general, cytochrome reductase expression levels in most primary cells are sufficient to allow a suitable enzymatic activity in cells transformed with the vectors herein described. However, if a cell line to be transformed by the inclusion of any sequence coding for a CYP enzyme does not show enough reductase activity, it can be co-infected simultaneously with two adenoviral vectors, the first one carrying the CYP sequence of interest, and the second one carrying the sequence of a CYP reductase, so that said cell line could be able to express both enzymes. An alternative to the latter is to include both genes in the same adenoviral construct in order to infect the cells with both genes at the same time.

### BACKGROUND OF THE INVENTION

Drug Metabolism, the Leading Cause of the Variability of Clinical Responses in Humans

It is known that drug metabolism is the leading cause of the variability of clinical responses in humans. Drugs, in addition to exerting a pharmacological action on a given target tissue, undergo chemical transformations during their transit through the organism (absorption, distribution and excretion). This process is known as drug metabolism or biotransformation, and can take place in all organs or tissues with which the drug is in contact. The process is catalysed by a group of enzymes generically known as drug metabolisation or biotransformation enzymes, mainly present in the microsomal and/or cytosolic cell fractions, and to a lesser extent in the extracellular space, which include various oxygenases, oxidases, hydrolases and conjugation enzymes (Garattini 1994). In this context, the liver ‘is the most relevant organ, and monooxygenases dependent on the P450 (CYP450) cytochrome together with flavin-monooxygenases, cytochrome C reductase, UDP-glucoronyl transferase and glutation transferase are the enzymes most directly involved (Watkins 1990). The intestine, lungs, skin and kidney follow in importance as regards their ability to metabolise xenobiotics (Krishna 1994). These biotransformation processes can also be performed by the saprophytic microorganisms colonising the intestinal tract.

The phenomenon of biotransformation is crucial in the context of drug bioavailability, variability of pharmacological response and toxicity, and understanding it is vital for an improved medicament use and development. In fact, biotransformation is the most variable stage and that which affects most the plasma drug levels after administration to various individuals. The rate at which a drug is biotransformed and the number and abundance of the various metabolites formed (metabolic profile) can vary greatly among individuals, explaining that for some a given drug dose can be therapeutically effective, as it generates adequate plasma levels, while for others it is ineffective as a faster metabolisation does not allow obtaining the therapeutic plasma concentration. The situation is even more serious in individuals lacking one of the enzymes involved in the drug metabolism, who attain plasma levels much higher than the expected levels after a dose that is tolerated well by the rest of the population (Meyer 1997).

Biotransformation Enzymes Present Geno/phenotypic Variability

The great variability in drug and xenobiotic metabolism among human population groups/individuals has been confirmed numerous times (Shimada et al 1994). Two factors are mainly responsible for these differences: the inducibility of biotransformation enzymes by xenobiotics and the existence of gene polymorphisms.

Indeed, one of the characteristics of biotransformation enzymes is that they can be induced by xenobiotics, so that exposure to these compounds results in a greater expression of the enzymes. Agents such as drugs, environmental pollutants, food additives, tobacco or alcohol act as enzyme inducers (Pelkonen et al 1998). A “classical” definition of induction involves synthesis de novo of the enzyme as a result of an increased transcription of the corresponding gene, as a response to an appropriate stimulus. However, in studies on xenobiotic metabolism this term is often used in a wider sense to describe an increase in the amount and/or activity of the enzyme due to the action of chemical agents, regardless of the mechanism causing it (such as increased transcription, stabilisation of mRNA, increased translation or stabilisation of the enzyme) (Lin and Lu 1998). The phenomenon of induction is not exclusive of the CYP and also affects conjugation enzymes. However, the induction processes that have been studied in greater depth are those affecting the CYP and the inducers are classified according to the CYP isoenzymes on which they can act (Pelkonen et al 1998, Lin and Lu 1998).

However, not all of these differences in the biotransformation activity can be attributed to the action of inducers. It has been verified that genetic factors, specifically gene polymorphisms, are also involved in this variability (Smith et al 1998). CYP isoenzymes (CYP1A1/2, 2A6, 2C9, 2C19, 2D6, 2E1) and conjugation enzymes (N-acetyltransferase and glutation S-transferase) are polymorphically expressed (Blum 1991, Miller et al 1997).

The gene polymorphism of P450, together with phenotypic variability, is the leading cause for interindividual differences in drug metabolism. This is due to the existence of genetic changes as a consequence of mutations, deletions and/or amplifications. Typically, there are two situations (Meyer y Zanger 1997): (i) subjects with defective genes (mutated, incomplete, inexistent, etc.) because of which they metabolise the drug poorly (slow metabolisers); and (ii) individuals with duplicated or amplified functional genes which thus show a greater metabolisation capacity (ultrafast metabolisers).

The most widely studied polymorphisms are those of debrisoquine/sparteine hydroxylase (CYP2D6) (Skoda 1988; Kimura et al. 1989; Heim y Meyer 1992), and S-mefenitoine hydrosylase (CYP2C19) (Wrighton et al. 1993; De Morais 1994; Goldstein et al 1994), which respectively affect over 7% and 5% of the Caucasian population, and which can produce significant alterations in the metabolisation of over 30 commonly-used drugs.

Clinical Relevance of Metabolic Variability and Idiosyncrasy

Drug metabolism by hepatic enzymes must be understood as a set of reactions in which various enzymes compete for a same substrate, the drug. The affinity of the drug for each enzyme (K_{M}) and the kinetic characteristics of the reaction catalysed by it (V_{MAX}) will determine the importance of the reaction in the overall context of the drug metabolism. Thus, two extreme situations may exist a) the compound is a substrate for various enzymes, yet originates basically one metabolite, or b) several enzymes are involved in its metabolism, resulting in various metabolites being produced.

In the first case, a different expression of the enzymes involved in the metabolism of a drug results in differences in its rate of metabolisation, and thus in its pharmacokinetics. This phenomenon can result on one hand in a deficient drug metabolisation, with the ensuing accumulation of the compound in the organism, abnormally high plasma levels and, on the other hand, in a metabolisation so accelerated that it is impossible to attain suitable therapeutic levels and the desired pharmacological effect.

In the second case, the metabolic profile of the drug will be clearly different; this is, the amount and relative proportion of the metabolites produced would be different. This can translate into a lower pharmacological effectiveness if the metabolite, and not the compound administered, is pharmacologically active, or in the case of producing abnormal amounts of a more toxic metabolite responsible for adverse effects.

The geno-phenotypic variability of CYP, in addition to being directly responsible for the pharmacokinetic differences (bioavailability, half-life, rate and extent of metabolisation, metabolic profile) and indirectly responsible for the pharmacodynamic differences (therapeutic ineffectiveness/exaggerated response, undesired effects) (Miller et al 1997, Smith et al 1998), lies at the root of idiosyncratic toxicity (Pain 1995). Oftentimes, during its metabolism the drug can give rise to another metabolite more toxic to the cell, or be converted into a more reactive chemical species that can interact with other biomolecules (bioactivation). This type of reactions, a relative exception for a substantial part of the population, can have a considerable importance in other individuals with singular expression levels of the various CYP's (Meyer 1992).

Models Used to Predict Effects Due to Changes in CYP Expression

The availability of in vitro systems that can faithfully reproduce the in vivo metabolism of drugs is one of the goals pursued by various research groups. The research group of the inventors has developed cultivation of human hepatocytes and their use in pharmaco-toxicologic studies (Bort et al 1996, Castell et al. 1997, Gómez-Lechón et al 1997). However, in these models it is only possible to affect the expression of biotransformation enzymes to a limited extent. For example, using enzymatic inducers it is possible to increase the expression levels of CYP's (Donato et al. 1995, Guillén et al. 1998, Li 1997). However, even using specific inducers such as methyl cholantrene, phenobarbital or rifampicine it is not possible to selectively modify one of them without affecting the others.

Another possible alternative is the use of genetically modified cell lines to overexpress one of the human CYPs (Bort et al. 1999a). While these lines are a useful tool in determining whether a specific enzyme is involved in the formation of a given compound, they do not allow discovering the extent to which differences in expression of a biotransformation enzyme affect a drug's metabolic profile and rate of a metabolisation by hepatocytes.

Possible Strategies for the at-will Modulation of the Expression of Cytochrome P450 (CYP 450) in Hepatocytes

The ideal model would be one allowing to modulate in a simple manner the individualised expression of an enzyme without affecting the others. In the case of induction, there are several experimental strategies that could be applied, based on the use of expression vectors with a promoter that can be activated by a specific exogenous compound in a concentration-dependent manner. In this way, depending on the activator concentration there will be a greater or lesser expression of the heterologous gene cloned “in phase” after the promoter. Among the various systems used, the following may be remarked:
a) the system based on operon Tn10a (Tet-on and Tet-off) (Gossen et al 1992, 1995; Resnitzky et al 1994) which requires a stable double transfection of the cells. There are two variants: Tet-on and Tet-off In the “Tet-on” system the cells are initially transfected with the “pTet-on” vector (resistance to G418), which allows a constitutional expression of the tTA hybrid protein, which is incapable of binding to the TRE-CMV promoter unless it has been previously joined to tetracycline. The second stable transfection is made with the pTRE vector (resistance to hygromycin) which contains an expression cassette with the TRE-CMV promoter. The ectopic gene is cloned in this vector. In the absence of tetracycline there is no expression of the ectopic gene. When tetracycline is added, and in a dose-dependent manner, it binds to the tTA protein allowing it to bind to the TRE-CMV promoter and thus allowing the expression of the protein. On its part, the “Tet-off” system consists of a first stable transfection with pTet-off (resistance to G418), which allows a constitutional expression of the tTA hybrid protein. This protein can bind to the TRE-CMV promoter, inducing expression of the “in-phase” protein. When it joins tetracycline it loses this capacity. The second stable transfection is made with the pTRE vector, which contains an expression cassette with the TRE-CMV promoter, in which the ectopic gene is cloned. In the absence of tetracycline a constitutional and high expression of the ectopic gene is obtained. When tetracycline is added, and in a dose-dependent manner, it binds to the tTA protein preventing its union to the promote and thus stopping the expression;
b) the GRE-ecdysone system (No et al 1996): this system also requires a double stable transfection of the cells. The first one uses the pVgRXR vector (resistance to zeocin) that constitutionally expresses the hybrid protein VgRXR. This protein cannot bind to the promoter regulated by glucocorticoids 5xE/GRE P_{^{□}}_{HSP} unless ecdysone has been previously bonded. A second transfection with pIND (resistance to G418) is used to introduce the ectopic gene in an expression cassette with the promoter 5xE/GRE P_{^{□}}_{HSP}. In the absence of ecdysone there is no expression of the ectopic gene. When ecdysone is added, in a dose-dependent manner, it binds to the VgRXR protein, allowing union to the 5xE/GRE P_{^{□}}_{HSP} promoter and thus the expression of the protein; and
c) systems based on the metallothionein promoter (Stuart et al. 1984). The metallothionein promoter presents a capacity to regulate the expression of the gene located “in phase” as a function of the doses of Zn²⁺ and other heavy metals. In the absence of Zn²⁺ there is no expression of the ectopic gene. When Zn²⁺ is added the gene expression increases in a dose-dependent manner.

There are several problems associated to the use of these expression vectors. Firstly, they are not strictly dose-dependent, and often behave in an all-or-nothing fashion, or are not fully blockable. In addition, in the case of Tet on/Tet off and Ecdysone two stable transfections are required, which in view of the extraordinary resistance of hepatocytes to transfections makes successful results highly unlikely. Because of this, nowadays there are no efficient cell models that can reproduce human variability of drug metabolism in vitro.

Thus, one aspect of this invention relates to a method for obtaining a singular ceil model that can reproduce the metabolic idiosyncrasy of humans in vitro. This method is based on the use of expression vectors that code for the sense and anti-sense mRNA of the enzymes of drug biotransformation Phases 1 and II. These expression vectors preferably contain ectopic DNA sequences that code for the sense and anti-sense mRNA of drug biotransformation Phases 1 and II that present a greatest variability in humans.

The method disclosed in this invention allows modulating or modifying (increasing or diminishing) the individualised expression of an enzyme in a simple manner without affecting other enzymes. A singular cell model such as the one taught by this invention can be used in drug development studies, specifically in the study of drug metabolism, potential idiosyncratic hepatotoxicity, medicament interactions, etc.

In another aspect, the invention relates to a kit comprising one or more expression vectors that code for the sense and anti-sense mRNA of the enzymes of drug biotransformation Phases 1 and II. This kit can be used to carry out the method for obtaining a singular cell mode capable of reproducing in vitro the metabolic idiosyncrasy of humans provided by this invention.

### BRIEF DESCRIPTION OF THE FIGURES

FIG. 1 illustrates the blocking of the expression of HNF4 by anti-sense RNA and repression of CYP2E1.

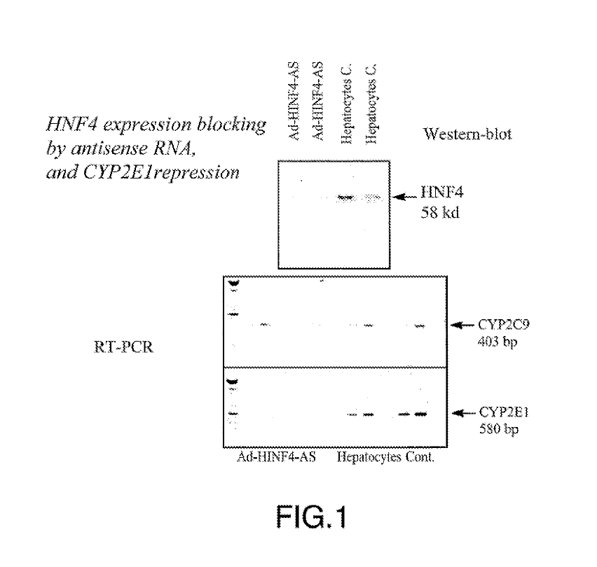

FIG. 2 is a bar chart showing the mRNA increase in HepG2I cells infected with different clones of the recombinant adenovirus identified as Ad-2E1.

FIG. 3 is a graph showing the increased activity in HepG2I cells infected with various concentrations of the recombinant adenovirus identified as Ad-3A4 and incubated with testosterone.

### DESCRIPTION OF THE INVENTION

In one aspect, the invention provides a method for obtaining a singular cell model capable of reproducing in vitro the metabolic idiosyncrasy of humans, wherein said model comprises a set of expression vectors that confer to the transformed cells a phenotypic profile of drug biotransformation enzymes designed at will, in order to reproduce the metabolic idiosyncrasy of humans, comprising:

c) Transforming cells expressing reductase activity with a set of expression vectors comprising ectopic DNA sequences that code for drug biotransformation enzymes selected from among Phase 1 drug biotransformation enzymes and Phase II drug biotransformation enzymes,

wherein each expression vector comprises an ectopic DNA sequence that codes for a different Phase 1 or Phase I I drug biotransformation enzyme selected from:

(iii) A DNA sequence transcribed in the sense mRNA of a Phase 1 or Phase II drug biotransformation enzyme (sense vector) and

(iv) a DNA sequence transcribed in the anti-sense mRNA of a Phase 1 or Phase II drug biotransformation enzyme (anti-sense vector);

wherein the expression of said ectopic DNA sequences in the cells transformed with said expression vectors confers to the transformed cells certain phenotypic profiles of the Phase 1 or Phase II drug biotransformation enzymes,

to obtain with said expression vectors cells that transitorily express said ectopic DNA sequences and present a different phenotypic profile of Phase 1 or Phase II drug biotransformation enzymes;

d) building a singular cell model capable of reproducing in vitro the metabolic idiosyncrasy of humans from said transformed cells transformed with said set of expression vectors, both sense vectors and anti-sense vectors, so that the result is the expression of any phenotypic profile of Phase 1 or Phase II drug biotransformation enzyme desired.

According to the method provided by the invention, cells that express reductase activity are transformed using a set of expression vectors. The existence of this reductase activity, CYP-reductase, in the cells to be transformed is essential, as it is not present or is insufficient the CYP protein contained in the expression vector will be expressed, but although it is active it will not be able to participate in the drug oxidation reactions.

The NADPH-cytochrome P450 reductase activity can be easily measured in the cells by an assay comprising, for example, cultivating the cells in 3.5 cm plates and using them when they reach 80% confluence. The cells are detached from the plates with the aid of a spatula in 1 ml of 20 mM phosphate buffer solution (PBS, pH 7.4), they are sonicated for 10-20 seconds and the homogenised obtained is centrifuged at 9,000 g for 20 minutes at 4° C. The supernatant (S-9 fraction) is used to evaluate the enzymatic activity. For this a 50 μg aliquot of the S-9 fraction protein is taken and incubated in 1 ml of 0.1 M potassium phosphate buffer (pH 7.2) containing 0.1 μM EDTA, 50 μM potassium cyanide, 0.05 μM cytochrome c and 0.1 μM NADPH. The reduction rate of the cytochrome c is determined by a spectrophotometer at 550 nm. The enzymatic activity is calculated using the molar extinction coefficient of 20×10³ M×cm⁻¹, and the results are expressed as nmol of cytochrome c reduced per minute and per mg of cell protein.

Practically any cell expressing reductase activity can be used to carry out the method of the invention, such as a human or animal cell, including tumour cells. Preferably, said cell is a human cell selected from among cells of hepatic, epithelial, endothelial and gastrointestinal type CaCO-2 origin. In a specific embodiment, this human cell is a hepatocyte or a HepG21 cell. In another specific embodiment, the cell expressing reductase activity is a human or animal cell, including tumour cells which, lacking the Phase 1 or Phase II drug biotransformation enzyme, is infected with a combination of one or more of the expression vectors of the invention, containing each of these in a certain concentration so that a cell is generated with a metabolic capability similar, for example, to that of a hepatocyte, with a normal or singular phenotype.

The expression vectors used to transform these cells expressing reductase activity, hereinafter referred to as the expression vectors of the invention, comprise the ectopic DNA sequences coding for drug biotransformation enzymes selected from among the previously defined Phase 1 drug biotransformation enzymes and Phase II drug biotransformation enzyme. Illustrative examples of Phase 1 and Phase II drug biotransformation enzyme include various oxygenases, oxydases, hydrolases and conjugation enzymes, among which the monooxygenases dependent on CYP450, flavin-monooxygenases, sulfo-transferases, cytochrome C reductase, UDP-glucoronyl transferase, epoxide hydrolase and glutation transferase are enzymes greatly involved in drug biotransformation.

In general, each expression vector of the invention comprises an ectopic DNA sequence that codes for a different Phase 1 or Phase II drug biotransformation enzyme, selected from among the above-defined sequences (i) (sense) and (ii) (anti-sense). Any ectopic DNA sequence coding for a Phase 1 or Phase II drug biotransformation enzyme can be used to build the expression vectors of the invention. However, in a specific embodiment the ectopic DNA sequence coding for a Phase 1 or Phase II drug biotransformation enzyme is selected from the group formed by the DNA sequences transcribed in the sense mRNA or anti-sense mRNA of CYP450 isoenzymes, such as CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5 or GST(A1), and DNA sequences transcribed in the sense mRNA or anti-sense mRNA of enzymes such as oxygenases, oxydases, hydrolases and conjugation enzymes involved in drug biotransformation, such as DNA sequences transcribed in the sense mRNA or anti-sense mRNA of flavin-monooxygenases, sulfo-transferases, cytochrome C reductase, UDP-glucoronyl transferase, epoxide hydrolase or glutation transferase. The expression of these ectopic DNA sequences in the cells transformed with the expression vectors of the invention confers to said cells certain phenotypic profiles of Phase 1 or Phase II drug biotransformation enzymes.

In a specific embodiment, said ectopic DNA sequence coding for a Phase 1 or Phase II drug biotransformation enzyme is a DNA sequence transcribed in the sense mRNA of a Phase 1 or Phase II drug biotransformation enzyme.

In another specific embodiment, said DNA sequence coding for a Phase 1 or Phase II drug biotransformation enzyme is a DNA sequence transcribed in the anti-sense mRNA of a Phase 1 or Phase II drug biotransformation enzyme.

The gene expression regulation strategy using anti-sense technology mainly consists of inserting in a cell an RNA molecule or an oligodeoxynucleotide whose sequence is complementary to that of a native mRNA that one desires to block. The specific and selective bonding of these molecules prevents translation of the messenger and synthesis of the corresponding protein (Melton 1985, Stein and Cheng 1993, Branch 1998). The final result is the targeted inactivation of the expression of a selected gene. The success of this strategy depends on various factors that are technically difficult to achieve, such as having an efficient system to insert the anti-sense molecule in the cell interior, said molecule interacting specifically with the target mRNA and not with other mRNA's, and that it is resistant to cell degradation systems. The two most commonly used procedures involve the use of an expression vector that includes a cloned cDNA in an inverse position (Melton 1995); when this vector is transfected to the cell interior it expresses a non codifying RNA or RNA fragment (without sense) that will associate by specific base pairing with its complementary native mRNA, or instead the use of oligo phosphothiolates that are oligodeoxynucleotides modified to make them resistant to intracellular degradation (Stein and Cheng 1993). It entry in the cell interior is solved by endocytosis or picnocytosis. The specific union to the target mRNA is harder to predict, so that the ideal oligo to block a specific mRNA can only be empirically determined [the success of this methodology has been greatly limited by the very low efficiency of the usual transfection procedures (10%)].

In a specific embodiment of the method provided by the present invention, recombinant adenoviruses have been built that can be used as carriers of a cDNA cloned with an inverted orientation as a source of antisense mRNA inside the cell. As the transfection efficiency is very high, about 100%, the “antisense” molecule is expressed in a very efficient manner in almost all target cells. The simplicity of the infection process in hepatocytes, which on another hand are very resistant to classical transfection techniques, makes this the model of choice. The viability of the proposed strategy is backed by recent results obtained by the inventors developing an adenovirus that codes for the anti-sense mRNA of the hepatic transcription factor HNF4. Transfection of human hepatocytes with this anti-sense adenovirus translates into the complete disappearance of the transcription factor HNF4 after 72 hours, as shown by the western-blot analysis. The protein most homologous to HNF4 is another transcription factor of the same family known as RXRa. This protein does not undergo changes, thereby showing that the anti-sense blocking is completely specific. The targeted inactivation of this transcription factor led to the loss of expression of certain CYP's, specifically CYP2E1.

Almost any system for transferring DNA exogenous to a cell can be used to build the expression vectors of the invention. In a specific embodiment, the expression vector of the invention is selected from among a viral vector, a liposome or a micellar vehicle, such as a liposome or micellar vehicle useful for gene therapy. In general, any virus or viral vector capable of infecting the cells used to put in practice the method of this invention can be used to build the expression vector of the invention. Advantageously, expression vectors will be chosen that can express transgenes in a highly efficient and quick manner in the transformed cells. In a specific embodiment, this virus is a natural or recombinant adenovirus, or a variant of it, such as a type 5 subgroup C adenovirus. The adenovirus is a non-oncogenic virus of the Mastadenoviridae genus, whose genetic information consists of a double linear DNA chain of 36 kilobases (kb) divided into 100 mu (map units; 1 mu=360 pb). Information on its replicative cycle has been provided by Greber 1993, Ginsberg 1984 and Grand 1987.

The adenovirus easily infects many cell types, including hepatocytes, so that they are a useful tool for transfecting exogenous genes to mammal cells. Specifically, the adenovirus is an excellent expression vector that has the additional advantage of showing a very high efficiency for hepatocyte transfection (equal to or greater than 95%). Additionally, the expression degree is proportional to the infective viral load and, finally, the transgene expression does not affect the expression of other hepatic genes (Castell et al. 1998).

Introduction of ectopic genes in the DNA of an adenovirus is limited by two facts: (i) the virus cannot encapsulate more than 38 kb (Jones 1978 and Ghosh Choudhury 1987); and (ii) its large size hinder cloning as unique restriction points are infrequent. To solve these problems, several strategies have been employed, the most widely used of which is that developed by McGrory et al. 1988 or homologous recombination. In short, the procedure essentially consists of using two plasmids, pJM17 and pACCMV, which contain a homologous fragment of the incomplete adenovirus sequence. Its homologous nature allows the recombination of the two plasmids, resulting in a defective (non replicative) virus in whose genome is the gene that must be expressed. Plasmid pJM17, developed by McGrory et al. 1988, is a large plasmid (40.3 kb) that contains the complete circularized genome of the type 5 adenovirus dl309 (Jones 1978) which has the plasmid pBRX (ori, ampᵣ and tetᵣ) in its locus Xbal in 3.7 mu. Although pJM17 contains all the necessary information for generating infective viruses, its size exceeds the encapsulation size so that it cannot generate new virions. In order for the adenovirus generated after recombination to be capable of reproducing, co-transfection is performed in the human embryonic cell line of renal origin 293 (ATCC CRL 1573) that expresses the region E1A of the type 5 adenovirus (Graham 1977). In this way, the supply of the protein E1A, a transcription factor acting in trans, by the host cell allows multiplying the recombinant virus inside it. It must be remarked that for its replication in the line 293 the recombinant virus also needs certain subregions of El in cys. These are the subregion lying between 0 and 1.3 mu, and that between 9.7 mu and the end of El. Between 0 and 0.28 mu is the ITR (internal terminal repeats) with the replication origin, between 0.54 and 0.83 the packing signals (Hearing 1987) and lastly, after 9.7 mu, is a segment surrounding the gene of protein IX. For this reason these regions are maintained in pACCMV, in which only 3 kb have been eliminated from the El region to make room for the expression module, without preventing the normal replication of the virus in 293.

Example 1 shows how to obtain recombinant adenoviruses containing ectopic DNA sequences that are transcribed in the sense mRNA or antisense mRNA of CYP450 isoenzymes, such as CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5 or GST(A1). These recombinant adenoviruses can be used to transform (infect or transfect) cells expressing reductase activity, for example, cells of hepatic origin such as HepG2l.

One characteristic of the method provided by this invention lies in its versatility for generating singular cell models with specific phenotypes by only varying the concentrations of the expression vectors of the invention used to transform said cells. In fact, it is possible to obtain models that allow comparing the metabolism of a drug in a liver with 10 3A4 and 1 2D6 with respect to another with 1 3A4 and 10 2D6, for example, by simply changing the types and amounts of expression vectors of the invention to be used to transform the cells. Tests conducted by the inventors have revealed that the response of this model is practically linear, this is, the greater the amount of expression vector of the invention the more activity is expressed, up to a limit (when cytopathic effects appear in the cells). Several tests have revealed that, depending on the expression vector of the invention used, up to about 300 CFU (colony forming units) there are no significant alterations in any other function of the cells (human hepatocytes) transformed by said vectors.

Transformation of the cells with the expression vector of the invention can be performed by any conventional method for transferring DNA exogenous to a cell, such as infection or transfection, depending among other factors of the expression vector of the invention employed. In a specific embodiment, the expression vectors of the invention used are recombinant adenoviruses and the cells can be transformed by infection, for which the cells must be at 70% confluence. In short, the culture medium maintaining the cells is aspirated and the latter are washed with a base medium or saline buffer; two washes of 2 or 3 ml each shall be performed. The amount of virus to be used may vary, according to the amount of activity desired to be expressed by the cells and their susceptibility. The adenovirus is diluted in the culture medium until the concentration reaches the range of 1 to 50 MOI (multiplicity of infection). The volume of culture used to maintain the cells will depend on the size of the plate, the final infection volume will be reduced to ¼ of the initial volume. The incubation time will be between 1 hour 30 minutes and 2 hours, at 37° C. The activity of the transgene in the infected cells can be detected after 24 hours, reaching a maximum after 48 hours, depending on the cell used. The total maximum amount of virus that a specific cell will admit is limited. This amount is determined by adding increasingly large amounts of virus until apparent cytotoxic effects are observed (morphology, cell function). This allows establishing the maximum number of viral particles that a specific cell will tolerate. The expression vectors of the invention can be used to transform transitorily the cells expressing reductase activity. This transitory transformation will be designed a priori to obtain the desired balance of expression of Phase 1 and Phase II drug biotransformation enzyme, in order to limit individual variability (metabolic idiosyncrasy), especially marked in the CYP system of humans. The combined use of variable amounts of different expression vectors of the invention (for example, some could express a Phase 1 or Phase II drug biotransformation enzyme and other their anti-sense mRNA) permits the necessary modulation, being established a priori, taking as a limit the viral load tolerated by each cell system.

Therefore, the invention constitutes a first approach based on the use of expression vectors, both sense and anti-sense, in a controlled manner, to modulate (increase or decrease) each of the Phase 1 or Phase II drug biotransformation enzyme in cells expressing reductase activity transformed by said vectors, so that these cells can reproduce at will a specific phenotype and provide an in vitro model for any conceivable human phenotypic profile, in a sample manner by only adding a controlled amount of expression vector to said cells.

A considerable share of the problems arising in medicament use (unexpected undesirable effects, lack or excessive therapeutic activity for the same compound dose, etc.) are greatly due to the fact that humans do not metabolise drugs identically. Thus, the same dose can lead to different plasma levels in different individuals, and/or metabolise to give a different metabolite profile in different persons. It is often the case that because of the greater or lesser presence of a specific biotransformation enzyme, the hepatic metabolites produced (or their relative proportion) can be remarkably different. Occasionally, low levels of enzymes whose action results into production of low toxicity metabolite(s), is poorly expressed in a given individual, so that metabolism of the drug in this individual will follow alternative paths that may produce much more toxic metabolites which are a minority in other individuals. In other cases it can be the abnormally high presence of a given enzyme, minoritary in other individuals, that leads to the production of a more toxic metabolite. These differences (metabolic idiosyncrasy) are an added risk factor in the arduous task of making a molecule become a new medicament. The reason for this is simple: compounds that have not shown adverse effects in the first clinical assays may, when widening their use to a greater population, allowing entry of individuals with metabolic singularities, produce idiosyncratic toxicity effects that can cause the financial failure of the development.

The present invention allows manipulating at will the levels of the various drug biotransformation enzymes of a human cell, as occurs in humans, to study in the cell whether the singularity can be relevant in a generalised clinical use of a new compound.

Therefore, in another aspect, the invention relates to the use of expression vectors (sense or anti-sense) of Phase 1 or Phase II drug biotransformation enzymes in the manipulation of cells, such as human and animal cells, including tumour cells, in order to reproduce in these cells the metabolic variability occurring in humans. Said vectors allow modifying at will the expression of a given enzyme without affecting the others. In this way it is possible to manipulate cells making them express the amounts of each enzyme desired (as viral vectors can be used alone or in combination), thereby simulating the variability that occurs in humans. The present invention allows studying and anticipating the possible relevance for a person of different expression levels of drug biotransformation enzyme when administering a new drug, before it is used in humans, thereby constituting an experimental singular cell model allowing to simulate or reproduce in vitro the variability existing in humans. In addition, the invention allows predicting the consequences of the different expression of drug biotransformation enzymes on the metabolism, pharmacokinetics and potential hepatotoxicity of a drug in process of development.

In another aspect, the invention relates to a kit comprising one or more expression vectors coding for the sense and anti-sense mRNA of Phase 1 and Phase II drug biotransformation enzymes. This kit can be used to put in practice the method for obtaining a singular cell model capable of reproducing in vitro the metabolic idiosyncrasy of humans provided by this invention.

### EXAMPLE 1

### Generation of Recombinant Adenoviruses

Cloning of Various Human Biotransformation Enzymes from an Own Human Liver Bank

The strategy used for cloning human CYP biotransformation enzymes 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5 or GST(A1) was performing a high-fidelity RT-PCR on a library of human hepatic cDNA's using primer oligonucleotides that flank the sequences coding for such enzymes.

The reaction mixture for reverse transcriptase (RT) consisted of 20 μl 1× reverse transcriptase buffer, DTT 10 mM, dNTPs 500 μM, 3 μM primer oligo d(T), 14, 60 U Rnase OUT and 250 U Rtase H. To this mixture was added 1 μg of total RNA. The reaction was performed for 60 minutes at 42° C., followed by heating for 5 minutes at 95° C. and a quick cooling in ice. The cDNA was stored at −20° C. until it was used.

### Primer Oligonucleotides Used

For each CYP two pairs of primer oligonucleotides flanking their coding sequence were designed. Each primer contains an additional sequence in the 5′ end corresponding to a restriction site for a specific enzyme, wherein they will be cloned in the pACCMV vector [see Table 1]

TABLE 1
Primer oligonucleotides used to clone the genes

| | | Fragments | Melting | Page |
|---|---|---|---|---|
| Oligonucleotides | Sequences 5′ to 3′ | (pb) | T (° C.) | no. |
| CYP 1A1 FP | cctccaggatccctacactgatc | | | |
| | (SEQ ID NO: 1) | | | |
| CYP 1A1 RP | cccggatcccagatagcaaaac | | | |
| | (SEQ ID NO: 2) | | | |
| CYP 1A2 FP | gcaggtaccgttggtaaagatggcatt | 1596 | 62.0 | M1433 |
| | (SEQ ID NO: 3) | | | 7 |
| CYP 1A2 RP | agccatggaccggagtcttaccaccac | | 60.8 | |
| | (SEQ ID NO: 4) | | | |
| CYP 2A6 FP | cccgaattcaccatgctggcctcagg | 1531 | 64.0 | X1393 |
| | (SEQ ID NO: 5) | | | 0 |
| CYP 2A6 RP | ccgaattccagacctgcaccggcaca | | | |
| | (SEQ ID NO: 6) | | | |
| CYP 2B6 FP | cagggatcccagaccaggaccatggaa | 1482 | 62.7 | M2987 |
| | (SEQ ID NO: 7) | | | 4 |
| CYP 2B6 RP | tttgggatccttccctcagccccttcag | | | |
| | (SEQ ID NO: 8) | | | |
| CYP 2C8 FP | ggggtaccttcaatggaaccttttgtgg | 1515 | | Y0049 |
| | (SEQ ID NO: 9) | | | 8 |
| CYP 2C8 RP | cccaagcttgcattcttcagacaggg | | | |
| | (SEQ ID NO: 10) | | | |
| CYP 2C9 RP | ggaattcggcttcaatggattctcttgtgg | 1485 | | M6185 |
| | (SEQ ID NO: 11) | | | 5 |
| CYP 2C9 FP | cgtctagacttcttcagacaggaatgaa | | | |
| | (SEQ ID NO: 12) | | | |
| CYP 2C18 FP | cccgaattcaccatgctggcctcagg | 1515 | | M6185 |
| | (SEQ ID NO: 13) | | | 3 |
| CYP 2C18 RP | ccgaattccagacctgcaccggcaca | | | |
| | (SEQ ID NO: 14) | | | |
| CYP 2C19 FP | atggatccttttgtggtcctt | | | M6185 |
| | (SEQ ID NO: 15) | | | 4 |
| CYP 2C19 | agcagccagaccatctgtg | | | |
| RP | (SEQ ID NO: 16) | | | |
| CYP 2D6 FP | ctaagggaacgacactcatcac | | | |
| | (SEQ ID NO: 17) | | | |
| CYP 2D6 RP | ctcaccaggaaagcaaagacac | | | |
| | (SEQ ID NO: 18) | | | |
| CYP 2E1 FP | | 1649 | | J0262 |
| | | | | 5 |
| CYP 2E1 RP | | | | |
| CYP 3A4 FP | | 1602 | | M1890 |
| | | | | 7 |
| CYP 3A4 RP | | | | |
| CYP 3A5 FP | gttgaagaatccaagtggcgatggac | 1707 | 58.3 | J0481 |
| | (SEQ ID NO: 19) | | | 3 |
| CYP 3A5 RP | acagaatccttgaagaccaaagtagaa | | 53.0 | |
| | (SEQ ID NO: 20) | | | |
| GST(A1) FP | ccaggatcctgctatcatggcagagaa | 735 | 50.9 | M2175 |
| | (SEQ ID NO: 21) | | | 8 |
| GST(A1) RP | tatggatcccaaaactttagaacattggtattg | | 47.9 | |
| | (SEQ ID NO: 22) | | | |

### High Fidelity PCR

The newly synthesised cDNA is used to conduct a conventional PCR. The PCR reaction was conducted in a thermocycler with the following reaction mixture: 3 μl of cDNA ( 1/10 RT), 3 μl buffer (10×), 50 μM dNTPs, 1 U total High Fidelity (Roche), 6 μM primer oligonucleotides and water to a final volume of 30 μl. The program used in the thermocycler consisted of:

A) Initial denaturalisation: 3 minutes at 95° C.

B) 4 cycles of:

d.—denaturalisation by cycles: 40 s at 95° C.

e.—ringing: 45 s at 58° C. (different for each primer)

f.—final elongation: 5 minutes at 74° C.

C) 30 cycles (more specific) of:

d.—denaturalisation by cycles: 40 s at 95° C.

e.—ringing 45 s at 62° C. (different for each primer)

f.—followed by a final elongation of 5 minutes at 74° C.

The product amplified by PCR was purified by column chromatography (High pure PCR product purification kit) and eluted by TE buffer. Then the PCR products were analysed by electrophoresis in 1.5% agarose gel and visualised with ethidium bromide to confirm the sizes of the amplified cDNA's.

Characterisation of the Cloned Genes. Digestions with Restriction Enzymes. Agarose Gels. Sequentiation

Prior to cloning the DNA was incubated with restriction enzymes in the buffer recommended by the manufacturer. A standard incubation mixture must include: 2 units of enzyme/μg of DNA, 10× buffer and distilled water. Occasionally, some enzymes require 100 μg/ml BSA or are incubated at 25° C.

Generation of pACCCMV Recombinant Plasmids

Subcloning of cDNA fragments (insert) in a pACCMV vector (vector) was performed by ligation of cohesive ends with the same restriction enzyme. This strategy produces clones with a sense and anti-sense orientation. In addition to the ligation itself, it includes prior dephosphorilation steps of the vector ends to prevent their recircularisation, for which added to the previous tube were 2 μl of CIP (20-30 U/μl Gibco BRL cat n° 18009019) and it was incubated for 20 minutes at 37° C. Then another 2 μl of CIP are added and it was incubated for 20 minutes at 56° C. To inactivate the enzyme and stop the reaction it was incubated for 10 minutes at 75° C.

Before ligation, the vector and the insert must be purified to eliminate remains of nucleotides, enzymes and buffers that may hinder the ligation. For this, the Geneaclean kit (Bio 101 cat n° 1001-200) is used to purify bands of a TAE-agarose gel (1% agarose in Tris-acetate 40 mM and EDTA 2 mM).

After purifying both bands the following reaction mixture was prepared for ligation:

2 μl vector (0.75 μg/μl)
4 μl insert (1 μg/μl)
1 111 T4 Ligase (1 U/μl) (Gibco BRL cat n° 15224-017)
1.5 μl 10x buffer
6.5 μl water
15.0 μl total

In parallel, a control mixture without insert was prepared. After 2 hours at ambient temperature competing bacteria were transformed with the ligation mixtures.

Ligation of cohesive ends was performed with the following reaction mixture:
1 μl vector (0.5 μg/μl)
4 μl insert (1 μg/μl)
1 μl T4 Ligase (1 U/μl) (Gibco BRL cat n° 15224-017)
1.5 μl 10×buffer
10.0 μl water

In parallel, a control mixture without insert was prepared. After 2 hours at ambient temperature competing bacteria were transformed with the ligation mixtures.

### Amplification of the Plasmids in Bacteria

Bacteria were used that had been previously treated with cold CaCl₂ solutions and subjected for a very short time to 42° C. to make them competing and receptive to the plasmid DNA: For this, 0.1-1 μg cDNA were added (ligation) to 100 μl of competing bacteria, the mixture was left in ice for 30 minutes and it was incubated in 1 ml of S.O.C. medium (Gibco BRL cat n° 15544-0189). Then 100 μl were transferred to an LB-agar medium plate with ampycillin (100 μg/ml) and it was left overnight at 37° C.

After this the bacteria were allowed to grow and they were used to amplify and purify the plasmid DNA by the procedure described hereinafter. An isolated colony of transformed bacteria is grown in 2-5 ml of LB medium with ampycillin. Then it is centrifuged at 8,000 rpm for 1 minute and the precipitate is resuspended in a lysis buffer (glucose 50 mM, Tris-HCl 25 mM, ph 8.0, EDTA and 4 mg/ml of lysozime). The suspension is left on ice for 5 minutes and it centrifuged at 10,000 rpm for 5 minutes. The supernatant is transferred to a clean tube, 500 μi isopropanol are added and it is centrifuged at 15000 rpm for 10 minutes. The supernatant is removed and the residue is washed with 70% ethanol (v/v), dried and resuspended in a suitable volume of TE pH 7.5 (Tris 10 mM, EDTA 1 mM).

After verifying the adequate colony with the restriction enzymes, the rest of the culture is transferred to a flask with 250 ml and it is grown overnight to amplify the plasmid.

Conventional kits were used to purify the plasmid DNA of the bacteria culture (between 250 and 500 ml).

Generation of the Adenovirus. Co-transfection of pJM17 and pAC-CYP Plasmids in 293 Cells

Co-transfection of the plasmids is performed in the 293 cell line, in which the recombinant virus generated by homologous recombination is able to replicate.

Co-transfection of the plasmids was performed by the calcium phosphate method, using different proportions. For this several plates of 6 cm diameter are seeded at 50-60% confluence. The next day tubes are prepared containing the different plasmids and/or carriers as well as the controls, and the content of each tube is added dropwise to 500 pl of HBS 2× (Hepes 50 mM, NaCl 140 mM, KCl 5 mM, glucose 10 mM and Na₂HPO₄ 1.4 mM adjusting to pH 7.15) and it is left for 20 minutes at ambient temperature. Then it is poured gently on the cell monolayer avoiding detachment, it is left for 15 minutes at ambient temperature, 4 ml of medium with serum are added, it is incubated in an oven at 37° C. for 4-6 hours, the medium is removed from the plates, 1 ml of medium without serum or antibiotics is added with 15% glycerol, 90 seconds are allowed to elapse and 5 ml PBS are added. Then it is washed twice with PBS to remove the glycerol completely, 5 ml of medium are added and it is stored in an oven, changing the medium every 3-4 days until cell lysis is observed.

After the recombination process occurs the virus will replicate in the 293 cells, managing to produce lysis in them (from 2 to 6 days). Then the virus is cloned, for which in plates covered with semisolid agar seriated 1/10-1/100 dilutions of the virus to be cloned are prepared in DMEM and 0.5 ml of each dilution are added to a 6 cm diameter plate with 293 cells, and the cells are incubated in an oven at 37° C. for 1 hour, shaking them for every 15 minutes. Then the medium is removed and the monolayer is covered with 6 ml of a mixture of agar 1.3% MEM 2× (1:1 v/v) previously heated to 45° C. and it is incubated in an oven at 37° C. After 7-9 days bald patches are visible, or areas in which the 293 cell monolayer is altered. These bald patches are selected and amplified in new plates of 293 cells.

Adenovirus Purification by Precipitation with PEG8000

A stock of pure virus was prepared by centrifugation in a CsCl gradient (method A) and, alternatively, using polyethylene glycol (method B), a simple method yielding similar results.

Method A

When the 293 cells undergo lysis the supernatant is removed and they are collected in PBS with MgCl₂ 1 mM, and 0.1% Nonidet p40.

Method B

In this case the cells have already undergone lysis and thus it is not possible to remove the medium. Nonidet p40 is added until it is left at 0.1%. It is then shaken for 10 minutes at ambient temperature and centrifuged at 20,000 g for 10 minutes. The supernatant is transferred to a clean tube and 0.5V are added of 20% PEG-8000/NaCl 2.5M, and it is incubated with shaking for 1 hour at 4° C. It is then centrifuged at 12,000 g for 10 minutes and the precipitate is resuspended in 1/100 to 1/50 of the initial medium volume in the following buffer: NaCl 135 mM, KCl 5 mM, MgCl₂ 1 mM and Tris-HCl 10 mM pH 7.4. Then it was dialysed overnight at 4° C. with the same buffer and filtered through a 0.22 μm filter to sterilise the stock. Finally, aliquots were obtained and conserved at −70° C. with 100 μg/ml de BESA.

Following the above procedure, recombinant adenoviruses were generated containing the DNA sequences coding for the CYP biotransformation enzymes CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5 or GST(A1). These recombinant adenoviruses (expression vectors of the invention) were named with the prefix “Ad” (adenovirus) followed by the name of the enzyme, this is, Ad-1A1, Ad-1A2, Ad-2A6, Ad-2B6, Ad-2C8, Ad-2C9, Ad-2C18, Ad-2C19, Ad-2D6, Ad-2E1, Ad-3A4, Ad-3A5 and Ad-GST(A1) respectively.

### EXAMPLE 2

### Transformation of Cells Expressing C Reductase Cytochrome Activity with Recombinant Adenoviruses

The recombinant adenoviruses obtained in Example 1 [Ad-1A1, Ad-1A2, Ad-2A6, Ad-2B6, Ad-2C8, Ad-2C9, Ad-2C18, Ad-2C19, Ad-2D6, Ad-2E1, Ad-3A4, Ad-3A5 and Ad-GST(A1)] were used to transform HepG2l cells by infection.

The culture medium containing a culture of HepG2l cells at 70% confluence was aspirated. The cells were washed twice with 2-3 ml of base medium or saline buffer each time. The amount of virus used was varied widely in order to generate a singular cell model encompassing a wide spectrum of human metabolic variability. The adenoviruses were diluted in the culture medium until reaching a concentration from 1 to 50 MOI. The volume of medium used to maintain the cells depends on the plate size, the final infection volume will be reduced to ¼ of the usual volume. The incubation time was kept from 1 hour 30 minutes to 2 hours at 37° C. The activity of the transgene in the infected cells can be detected after 24 hours, reaching a maximum after 48 hours, depending on the cell used. The maximum amount of total viruses admitted by a given cell is limited. To determine this amount increasingly large amounts of virus are added until apparent cytotoxic effects (morphology, cell function) are observed In this way it has been possible to establish the maximum number of viral particles tolerated by a given cell.

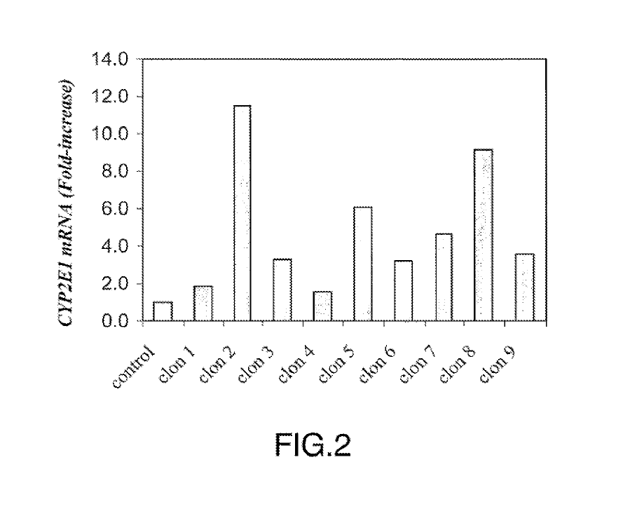

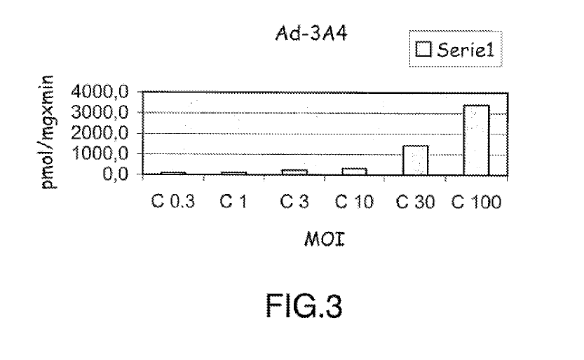

FIGS. 2 and 3 show specific examples of how it is possible to modify at will the expression of human enzymes relevant to drug metabolism. Specifically, FIG. 2 shows the increase of mRNA in HepG2l cells infected with various clones of Ad-2E1, while FIG. 3 shows the increased activity in HepG2l cells infected with various concentrations of Ad-3A4 and incubated with testosterone.

### BIBLIOGRAPHY

Andersson, T., Miners, J. O., Veronese, M. E., Tassaneeyakul, W., Tassaneeyakul, W., Meyer, U. A. y Birkett, D. J. (1993) Identification of human liver cytochrome P450 isoforms medianting omeprazole metabolism Br. J. Clin. Pharmacol. 36: 521-530.
Banks A. T., Zimmerman H. J. y Harter J. G. (1995) Diclofenac-associated hepatotoxicity analysis of 180 cases reported in the Food and Drug Administration as adverse reactions. Hepatology 22: 849-871
Bort R, Ponsoda, X., Carrasco, E., Gómez-Lechón M. J. y Castell, J. V. (1996) Metabolism of aceclofenac in humans. Drug. Met. Disp. 24: 834-851
Boelsterli U. A. (1993) Specific targets of covalent drug-protein interactions in hepatocytes and their toxicological significance in drug-induced liver injury. Drug Metaol. Rev 25: 395-451.
Bort, R., Macé, K., Boobis, A., Gómez-Lechón, M. J., Pfeifer, A. y Castell, J. V. (1999a), Hepatic metabolism of diclofenac. Role of human CYP in the minor oxidative pathways. Biochem. Pharmacol. en prensa.
Bort R., Ponsoda X., Jover, R., Gómez-Lechón, M. J. y Castell, J. V. (1999b) Diclofenac toxicity to hepatocytes: A role for drug metabolism in cell toxicity J. Pharmacol. Exp. Ther en prensa.
Blum, M., Demierre, A., Grant, D., Heim, M. y Meyer, U. (1991) Molecular mechanism of slow acetylation of drugs and carcinogens in humans. Proc. Natl. Acad. Sci. USA. 88: 5237-5241.
Branch A. D (1998) A good antisense molecule is hard to find. TIBS 23:45-50,
Castell, J. V., Hernández, D., Gómez-Foix, A. M., Guillén, 1., Donato, T. y Gómez-Lechón, M. J. (1997) Adenovirus-mediated gene transfer into human hepatocytes: analysis of the biochemical functionality of transduced cells. Gene Ther. 4: 455-464
Castell, J. V., Gómez-Lechón, M. J., Ponsoda, X. y Bort, R. (1997) The use of cultured hepatocytes to investigate the mechanisms of drug hepatotoxicity. Cell Biol. Toxicol. 13: 331-338.
Chardonnet, Y. y Dales, S. (1970) Early events in the interaction of adenoviruses with HeLa cells. 1. Penetration of type 5 and intracellular release of the DNA genome. Virology 40: 462-477.
Christians, U., Schmidt, G., Bader, A., Lampen, A., Schottmann, R., Linck, A. y Sewing, K. F. (1996) Identification of drugs inhibiting the in vitro metabolism of tacrolimus by human liver microsomes. Br. J. Clin. Pharmacol. 41: 187-190.
De Morais, S. M. F., Wilkinson, G. R. y Blaisdell, J. (1994) The major genetic defect responsible for the polymorfism of S-mephenytoin metabolism in humans. J. Biol. Chem. 269: 15419-15422.
Defer, C., Belin, M. T., Caillet Boudin, M. L. y Boulanger, P. (1990). Human adenovirus host interaction: comparative study with members of subgroups B and C. J. Viral. 67: 3661-3673
Donato, M. T., Gómez-Lechón, M. J. y Castell, J. V. (1993) A microassay for measuring cytochrome P4401A1 and P4501lB1 activities in intact human and rat hepatocytes cultured on 96-well plates. Anal. Biochem 213: 29-33.
Donato, M. T., Gómez-Lechón, M. J. y Castell, J V (1992) Biotransformation of drugs by cultured hepatocytes. En: In Vitro Alternatives to Animal Pharmaco-Toxicology. Eds J. V. Castell and M. J. Gómez-Lechón. Farmaindustria, Madrid, Chapter 7, pp. 149-178,
Donato M. T., J. V. Castell y M. J. Gómez-Lechón (1995) Effect of model inducers on cytochrome P450 activities of human hepatocytes in primary cultures. Drug Metab Disp 23: 553-558
Donato, M. T., Castell, J. V. y Gómez-Lechón, M. J. (1998) The coumarin 7-hydroxylation microassay in living hepatic cells in culture. ATLA 26: 213-233.
Fitzgerald, D. J. P., Padmanabhan, R., Pastin, P. y Willingham, M. C. (1983) Adenovirus induced release of epidermal growth factor and Pseudomonas toxin into the cytosol of KB cells during receptor mediated endocytosis. Cell 32: 607-617
Furukori, H., Otani, K., Yasui, N., Kondo, T., Kaneko, S., Shimoyama, R., Ohkubo, T., Nagasaki, T. y Sugawara, K. (1998) Effect of carbamazepine on the single oral dose pharmacokinetics of alprazolam. Neuropsychopharmacology 18: 364-369.
Garattini, S. (1994) Perspectives in the field of drug metabolism. Drug Metab. Rev. 26: 537-73
Ghosh-Choudhury, G., Haj Ahmad, Y. y Graham, F. L. (1987) Protein IX, a minor component of the human adenovirus capsid, is essential for the packaging of full length genomes. EMBO J. 6: 1733-1739
Gil, M. L., Ramirez, M. C, Terencio, M. C. y Castell, J. V. (1995) Immunochemical detection of protein adducts in cultured human hepatocytes exposed to diclofenac. Biochim. Biophys. Acta 1272: 140-146
Ginsberg, H. S (1984). The adenoviruses. Pienum Press, New York.
Gluzman, Y., Reichl, H. y Solnick, D. (1982) Helper free adenovirus type 5 vectors. En Eucaryotic vira) vectors (Y. Gluzman ed.) pp. 187-192. Cold Spring Harbor Laboratory Press, Cold Spring Harbor, New York.
Goldstein, J. A., Faletto, M. B., Romkes-Sparks, M., Sullivan, T., Kitareewan, S., Raucy, J. L., Lasker, J. M. y Ghanayem, B. I. (1994) Evidence that CYP2C19 is the major (S)-mephenytoin 4-hydroxylase in humans. Biochemistry 33: 1743-52.
Gómez-Foix, A. M., Coats, W. S., Baque, S., Alam, T., Gerard, R. D. y Newgard, C. B. (1992) Adenovirus mediated transfer of the muscle glycogen phosphorylase gene into hepatocytes confers altered regulation of glycogen metabolism. J. Biol. Chem. 267: 25129-25134.
Gómez-Lechón, M. J., Donato, T., Ponsoda, X., Fabra, R., Trullenque, R. y Castell, J. V. (1997) Isolation, culture and use of human hepatocytes in drug research. In In Vitro Methods in Pharmaceutical Research. J. V. Castell y M. J. Gómez-Lechón eds. pp. 129-154. Academic Press. London.
Gonzalez, F. J. y Gelboin, H. V. (1991) Human cytochromes P450: Evolution, catalytic activities and interindividual variations in expression. En: New Horizons in Biological Dosimetry. pp.11 20. Wiley Liss, Inc.
Gossen, M. y Bujark, H. (1992) Tight control of gene expression in mammalian cells by tetracycline-responsive promoters. Proc. Natl. Acad. Sci USA 89: 5547-5551.
Gossen, M., Freundlieb, S., Bender, G., Muller, G., Hillen, W. Y Bujard, H. (1995) Transcripcional activation by tetracyclines in mammalian cells Science 268: 1766-1769.
Graham, F. L., Smiley, J., Russell, W. C. y Nairn, R. (1977) Characteristics of a human cela line transformed by DNA from human adenovirus type 5. J. Gen. Virol. 36: 59-64.
Grand, R. J. The structure and functions of the adenovirus early region 1 proteins. Biochem. J. 241:25-38, 1987.
Greber, U. F., Willets, M., Webster, P. y Helenius, A. (1993) Stepwise dismantling of adenovirus 2 during entry into celas. Cell 75: 477-486,
Guillén 1., Donato, M. T., Jover, R., Castell, J. V., Fabra, R., Trullenque, R. y Gómez Lechón, M. J. (1998) Oncostatin M down-regulates basal and induced cytochromes P450 in human hepatocytes. J. Pharm. Exp. Ther.285:127-134.
Guillouzo, A. y Chesné, C. (1996) Xenobiotic metabolism in epithelial cell cultures. En Cell culture modeis in epithelial tissues: A practica) approach (Shaw A. J. ed.) pp. 67-85. Oxford, Oxford, U.K.
Hearing, P., Samulsky, R. J., Wishart, W. L. y Shenk (1987) Identification of a repeated sequence element required for efficient encapsulation of the adenovirus type 5 chromosome. J. Virol. 61: 2555-2558,
Heim, M. H. y Meyer, U. A. (1992) Evolution of a highly polymorphic human cytochrome P450 gene cluster: CYP2D6. Genomics 14: 49-58.
Helsby, N. A.; Ward, S. A.; Parslew, R. A. G.; Friedmann, P. S. y Rhodes, L. E. (1998) Hepatic cytochrome P450 CYP2C activity in psoriasis: studies using proaguanil as a probe compound. Acta Dermato Venereologica 78:81-83.
Jones, N. y Shenk, T. (1978) Isolation of deletion and substitution mutants of Adenovirus Type 5. Cell 13: 181-188.
Kimura, S., Umeno, M., Skoda, R. C., Meyer, U. A. y Gonzalez, F. J. (1989) The human debrisoquine 4-hydroxylase (CYP2D) locus: sequence and identification of the polymorphic CYP2D6 gene, a related gene, and a pseudogene. Am. J. Hum. Genet. 45:889-904.
Krishna, D. R. y Klotz, U. (1994) Extrahepatic metabolism of drugs in humans. Clin-Pharmacokinet 26:144-160.
Kronbach, T., Mathys, D., Gut, J., Catin, T. y Meyer, U. A. (1987) High-performance liquid chromatographic assays for bufuralol 1′-hydroxylase, debrisoquine 4-hydroxylase, and destromethorphan O-demethylase in microsomes and purified cytochrome P-450 isozymes of human liver. Analytical Biochemistry 162: 24-32
Lautenschlager, M. T., Viktor, S., Muller, U. A. y Hoffmann, A. (1996) Serum concentrations of caffeine, metamizol, ebrisoquine, sulfamethazine and their metabolites in diabetics before and during insulin therapy. Pharmazie 51: 750-753.
Lecoeur, S., Bonierbale, E., Challine, D., Gautier, J. C., Valadon, P., Dansette, P. M., Catinot, R., Ballet, R., Mansuy, D. y Beaune, P. H. (1994) Specificity of in vitro covalent binding of tienilic acid metabolites to human liver microsomes in relationship to the type of hepatotoxicity: comparison with two directly hepatotoxic drugs. Chem. Res. Toxicol. 7: 434 442.
Lewis, J. H. (1984) Hepatic toxicity of nonsteroidal anti-inflammatory drugs. Clin. Pharm. 3: 128-138
Li, A. P. (1997) Primary hepatocyte cultures as an in vitro experimental model for evaluation of pharmacokinetic drug-drug interactions. In Drug-Drug Interactions. Scientific and regulatory perspectives. A. P. Li, ed. pp. 103-130. Advances in Pharmacology, Academic Press. N.Y.
Lin, J. H. y Lu, Y. H. (1998) Inhibition and induction of cytochrome P450 and clinical implications. Clin. Pharmacokinet 35: 361-390.
McGrory, W. J., Bautista, D. y Graham, F. L. (1988) A simple technique for the rescue of early region 1 mutations into infectious human adenovirus type 5. Virology 163: 614 617.
Meeks, R. G., Harrison S. D. y Bull R. J. (1991). Hepatotoxicology. (R. G. Meeks, S. D. Harrison y R. J. Bull, eds.). CRC Press, Boca Raton, Fla.
Melton, D. A. (1985) Injected anti-sense RNAs specifically block messenger RNA translation in vivo. Proc. Natl. Acad. Sci. USA 82:144-148.
Meyer, U. A. (1992) Drugs in special patient groups: Clinical importance of genetics in drug effects. In Clinical Pharmacology Basic Principies of Therapeutics. K. F. Melmon y H. F. Morelli, eds. Pp. 875-894. McGra-Hill. N.Y.
Meyer, U. y Zanger, U. (1997) Molecular mechanisms of genetic polymorphisms of drug metabolism. Annu. Rev. Pharmacol. Toxicol 37: 269-96.
Miller, M. S., McCarver, D. G., Bell, D. A., Eaton, D. L. y Goldstein, J. A. (1997) Genetic
polymorphisms in human drug metabolic enzymes. Fundam. Appl Toxicol 40:1-14.
Miners, J. O. y Birkett, D. J. (1996) The Use of Caffeine as a Metabolic Probe for Human Drug-Metabolizing-Enzymes. General Pharmacol. 27:245-249.
Miners, J. O., Coulter, S., Tukey, R. H, Veronese, M. E. y Birkett, D. J. (1996) Cytochrome P450, 1A2, and 2C9 are responsible for the human hepatic O-demethylation of R- and S-naproxen. Biochem. Pharmacol. 51: 1003-1008, No, D., Yao, T. P. y Evans, R. M. Ecdysone-inducible gene expression in mammalian cells and transgenic mice. Proc. Natl. Acad. Sci USA 93: 3346-335.
Ourlin, J. C., Vilarem, M. J., Daujat, M., Harricane, M. C., Domergue, J., Joyeux, H., Baulieux, J. y Maurel, P. (1997) Lipid-mediated transfection of normal adult human hepatocytes in primary culture. Anal. Biochem. 247: 34-44.
Pain, A. J. (1995) Heterogeneity of cytochrome P450 ánd its toxicological significance. Hum. Exp. Toxicol. 14: 1 7.
Park, B. K. y Kitteringham, N. R. (1990) Drug protein conjugation and its immunological consequences. Drug Metab. Rev. 22: 87 144.
Pearce, R. E., McIntyre, C. J., Madan, A., Sanzgiri, U., Draper, A. J., Bullock, P. L., Cook, D. C., Burton, L. A., Latham, C. N. y Parkinson A. (1996) Effects of freezing, thawing, and storing human liver microsomes on cytochrome P450 activity. Archives of Biochemistry and Biophysics 331: 145-169.
Pelkonen, O., Maenpaa, J., Taavitsainen, P., Rautio, A. y Raunio, H. (1998) Inhibition and induction of human cytochrome P450 (CYP) enzymes. Xenobiotica 28: 1203-1253.
Peter, R., Bocker, R., Beaune, P. H., Iwasaki, M., Guengerich, F. P. y Yang, C. S. (1990) Hydroxylation of chlorzoxazone as a specific probe for human liver cytochrome P-45011E1. Chem. Res. Toxicol. 3: 566-573.
Philipson, L., Lonberg, K. y Petterson, U. (1968) Virus receptor interaction in an adenovirus system. J. Virol. 2: 1064-1075.
Ponsoda, X., Bort, R., Jover, R., Gómez-Lechón, M. J. y Castell, J. V. (1995) Molecular mechanisms of diclofenac hepatotoxicity: cell injury is associated to the metabolism of the drug and is preluded by a decrease in ATP levels. Toxicology in Vitro 9: 439-444.
Rahman, A., Korcekwa, R., Gorgan, J., Gonzalez, F. J. y Harris J. W. (1994) Selective biotransformation of Taxol to 6a-Hydroxytaxol by human cytochrome P450 2C8. Cancer Res. 54: 5543-5546.
Rasmussen, E., Eriksson, B., Oberg, K., Bondesson, U. y Rane, A. (1998) Selective effects of somatostatin analogs on human drug-metabolizing enzymes. Clin. Pharmacol. Ther. 64: 150-159.
Resnitzky, D., Gossenm, M., Bujard, H. y Reed, S. I. (1994) Acceleration of the GIIS phase transition by expression of cyclins D1 and E with an inducible system. Mol. Cell. Biol. 14: 1669-1679.
Sanwald, P., David, M. y Dow, J. (1996) Use of electrospray ionization liquid chromatography mass spectrometry to study the role of CYP2D6 in the in vitro metabolism of 5-hydroxtryptamine receptor antagonists. J. Chromatography 678: 53-61
Shimada, T., Yamazaki, H., Mimura, M., Inui, Y. y Guengerich, F. P. (1994) Interindividual variations in human liver cytochrome P-450 enzymes involved in the oxidation of drugs, carcinogens and toxic chemicais: Studies with liver microsomes of 30 Japanese and 30 Caucasians. J. Pharmacol. Exp. Ther. 270: 414-423.
Skoda, R. C., Gonzalez, F. J. y Demierre, A. (1988) Two mutant alleles of the human cytochrome P450 dbl gene (P450 IID1) associated with genetically deficient metabolism of debrisoquine and other drugs. Proc. Natl. Acad. Sci. USA 85: 5240-5243.
Smith, G., Stubbins, M. J., Harries, L. W. y Wolf, C. R. (1998) Molecular genetics of the human cytochrome P450 monooxygenase superfamily. Xenobiotica 28: 1129-1165.
Stricker, B. C. H. (1992) Drug-induced hepatic injury. 2a edición. Elsevier, Amsterdam, Holanda.
Stein, C. A. y Cheng, Y. C. (1993) Antisense oligonucleotides as therapeutic agents. Is the bullet really magical? Sience 261:1004-1012
Stuart, G. W., Searle, P. F., Chen, H. Y., Brinster, R. L. y Palmitér, R. D. (1984) A 12-base-pair DNA motif that is repeated severa) times in metallothionein gene promoters confers metal regulation to a heterologous gene. Proc. Natl. Acad. Sci. USA 81: 7318-7322.
Timbrell, J. A. (1991) Principies of biochemical toxicology. Taylor and Francis, London.
Transon, C., Lecoeur, S., Leemann, T., Beaune, P. y Dayer, P. (1996) Interindividual variability in catalytic activity and immunoreactivity of three major human liver cytochrome P450 isozymes. Eur. J. Clin. Pharmacol. 51: 79-85.
Trapnell, B. C. (1993) Adenoviral vectors for gene transfer. Adv. Drug Del. Rev. 12: 185-199.
Tsutsui, H., Terano, Y., Sakagami, C., Hasegawa, 1., Mizoguchi, Y. y Morisawa, S. (1992) Drug specific T cells derived from patients with drug induced allergic hepatitis. J. Immunol. 149:706 716.
Van Doren, K., Hanahan, D. y Gluzman, Y. (1984) Infection of eukaryotic cells by helper-independent recombinant adenoviruses: early region 1 is not obligatory for integration of viral DNA. J. Viro]. 50: 606-612.
Venkatakrishnan, K., Greenblatt, D. J., VonMoltke, L. L. y Shader, R. I. (1998) Alprazolam is another substrate for human cytochrome P450-3A isoforms. J. Clin. Psychopharmacol. 18: 256.
Wrighton, S. A., Stevens, J. C., Becker G. W. y VandenBranden, M. (1993) Isolation and characterization of human liver cytochrome P4502C19: correlation between 2C19 and S-mephenytoin 4′-hydroxylation. Arch. Biochem. Biophys. 306:240-245.
Watkins, P. B. (1990) Role of cytochromes P450 in drug metabolism and hepatotoxicity. Semin. LiverDis. 10: 235-250.
Yang, T. J., Shou, M., Korzekwa, K. R., Gonzalez, F. J., Gelboin, H. V. y Yang, S. K. (1998) Role of cDNA-expressed human cytochromes P450 in the metabolism of diazepam. Biochem. Pharmacol. 55: 889-896.
Zimmermann, H. J. (1982) Hepatotoxicity. Appleton Century Crofts Ed., New York. Zimmerman, H. J. y Maddrey, W. C (1995) Acetaminophen (paracetamol) hepatotoxicity with regular intake of alcohol: analysis of instances of therapeutic misadventure. Hepatology 22:767-773.

1. A method for obtaining a singular cell model capable of reproducing in vitro a metabolic idiosyncrasy of humans, wherein said model comprises one or more recombinant adenoviral expression vectors that confer to transformed cells a phenotypic profile of drug biotransformation enzymes designed at will, said method comprising:
a) Transforming human cells of hepatic origin expressing cytochrome P450 reductase activity with one or more expression vectors,
wherein each expression vector comprises an ectopic DNA sequence selected from the group consisting of:
(i) a DNA sequence transcribed in the sense mRNA of a Phase I or a Phase II drug biotransformation enzyme (“sense vector”); and
(ii) a DNA sequence transcribed in the anti-sense mRNA of a Phase I or a Phase II drug biotransformation enzyme (“anti-sense vector”);
wherein the expression of said ectopic DNA sequences in the cells transformed with one or more of the aforementioned expression vectors confers on the transformed cells specific phenotypic profiles of Phase I or Phase II drug biotransformation enzymes, and
b) building a singular cell model capable of reproducing in vitro the metabolic idiosyncrasy of humans from said cells transformed with the a forementioned set of expression vectors, both sense and anti-sense vectors, so that the result is the expression of any phenotypic profile of Phase I or Phase II drug biotransformation enzymes desired.

2. Method according to claim 1, wherein said Phase I and Phase II drug biotransformation enzymes are selected from the group consisting of oxygenases, oxydases, hydrolases and conjugation enzymes.

3. Method according to claim 1, wherein said Phase I and Phase II drug biotransformation enzymes are selected from the group consisting of monooxygenases dependent on CYP450, flavin-monooxygenases, sulfo-transferases, cytochrome C reductase, UDP-glucoronyl transferase, epoxide hydrolase and glutation transferase.

4. Method according to claim 1, wherein said ectopic DNA sequence coding for a Phase I or Phase II drug biotransformation enzyme is selected from the group consisting of: DNA sequences transcribed in the sense mRNA of CYP450 isoenzymes; anti-sense mRNA of CYP450 isoenzymes; DNA sequences transcribed in the sense mRNA of oxygenases, oxidases, hydrolases and conjugation enzymes involved in drug biotransformation; and DNA sequences transcribed in the anti-sense mRNA of oxygenases, oxidases, hydrolases and conjugation enzymes involved in drug biotransformation.

5. Method according to claim 1, wherein said ectopic DNA sequence coding for a Phase I or Phase II drug biotransformation enzyme is selected from the group consisting of: DNA sequences transcribed in the sense mRNA of CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5, GST(A1); DNA sequences transcribed in the anti-sense mRNA of CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5, GST(A1); DNA sequences transcribed in the sense mRNA of flavin-monooxygenases, sulfo-transferases, cytochrome C reductase, UDP-glucoronyl transferase, epoxide hydrolase and glutation transferase; and DNA sequences transcribed in the anti-sense mRNA of flavin-monooxygenases, sulfo-transferases, cytochrome C reductase, UDP-glucoronyl transferase, epoxide hydrolase and glutation transferase.

6. Method according to claim 1, wherein said ectopic DNA sequence coding for a Phase I or Phase II drug biotransformation enzyme is a DNA sequence transcribed in the sense mRNA of a Phase I or Phase II drug biotransformation enzyme.

7. Method according to claim 1, wherein said ectopic DNA sequence coding for a Phase I or Phase II drug biotransformation enzyme is a DNA sequence transcribed in the anti-sense mRNA of a Phase I or Phase II drug biotransformation enzyme.

8. Method according to claim 1, which comprises the combined use of variable amounts of said expression vectors comprising ectopic DNA sequences coding for the drug biotransformation enzymes selected from among Phase I drug biotransformation enzymes and Phase II drug biotransformation enzymes.

9. A human cell model capable of reproducing in vitro the metabolic idiosyncrasy of a human hepatocyte characterized in that said cells are human cells of hepatic origin expressing cytochrome P450 reductase activity, wherein said cells are transformed with one or more recombinant adenoviral expression vectors, wherein each expression vector comprises an ectopic DNA sequence that codes for a different Phase I or Phase II drug biotransformation enzyme, selected from among:
(i) a DNA sequence transcribed in the sense mRNA of a Phase I or Phase II drug biotransformation enzyme (“sense vector”); and
(ii) a DNA sequence transcribed in the anti-sense mRNA of a Phase I or Phase II drug biotransformation enzyme (“anti-sense vector”);
wherein the transitorily expression of said ectopic DNA sequences in the cells transformed with one or more of the aforementioned expression vectors confers the transformed cells specific phenotypic profiles of Phase I or Phase II drug biotransformation enzymes obtainable.

10. (canceled)

11. A method for studying the metabolism, pharmacokinetics, potential idiosyncratic hepatotoxicity, and/or potential medicament interactions of a drug, said method comprising placing said drug in contact with a singular cell model capable of reproducing in vitro the metabolic idiosyncrasy of humans obtained according to the method of claim 1.

12. (canceled)

13. A method to confer to any cell line of hepatic origin expressing cytochrome P450 reductase activity the capacity to metabolize xenobiotics in a controllable manner by means of one or more adenoviral expression vectors encoding Phase I enzymes and Phase II enzymes, said method comprising the transfection of said cells with one or more adenoviral expression vectors in order to confer to the transformed cells a phenotypic profile designed at will, up to metabolize xenobiotics, characterised in that the cell is transformed with a set of expression vectors comprising ectopic DNA sequences coding P450 enzymes involved in the xenobiotic biotransformation, wherein each expression vector comprises an ectopic DNA sequence transcribing for the sense mRNA of a different CYP enzyme, and wherein the expression of all of said ectopic sequences in the transformed cells confers to them a transitory xenobiotic metabolic profile.

14. Method according to claim 1, wherein said cell of hepatic origin expressing cytochrome P450 reductase activity is a human or animal cell, including tumour cells.

15. Method according to claim 1, wherein said adenoviral expression vectors are natural or recombinant adenoviruses.

16. The human cell model of claim 9, wherein said Phase I and Phase II drug biotransformation enzyme is selected from among oxygenases, oxydases, hydrolases and conjugation enzymes.

17. The human cell model of claim 9, wherein said Phase I and Phase II drug biotransformation enzyme is selected from among monooxygenases dependent on CYP450, flavin-monooxygenases, sulfo-transferases, UDP-glucoronyl transferase, epoxide hydrolase and glutation transferase.

18. The human cell model of claim 9, wherein said ectopic DNA sequence coding for a Phase I or Phase II drug biotransformation enzyme is selected from among the group of DNA sequences transcribed in the sense mRNA or anti-sense mRNA of CYP450 isoenzymes and DNA sequences transcribed in the sense mRNA or anti-sense mRNA of oxygenases, oxidases, hydrolases and conjugation enzymes involved in drug biotransformation.

19. The human cell model of claim 9, wherein said ectopic DNA sequence coding for a Phase I or Phase II drug biotransformation enzyme is selected from among the group of DNA sequences transcribed in the sense mRNA or anti-sense mRNA of CYP 1A1, CYP 1A2, CYP 2A6, CYP 2B6, CYP 2C8, CYP 2C9, CYP 2C18, CYP 2C19, CYP 2D6, CYP 2E1, CYP 3A4, CYP 3A5, GST(A1), and DNA sequences transcribed in the sense mRNA or anti-sense mRNA of flavin-monooxygenases, sulfo-transferases, UDP-glucoronyl transferase, epoxide hydrolase or glutation transferase.

20. The human cell model of claim 9, wherein said ectopic DNA sequence coding for a Phase I or Phase II drug biotransformation enzyme is a DNA sequence transcribed in the sense mRNA of a Phase I or Phase II drug biotransformation enzyme.

21. The human cell model of claim 9, wherein said ectopic DNA sequence coding for a Phase I or Phase II drug biotransformation enzyme is a DNA sequence transcribed in the anti-sense mRNA of a Phase I or Phase II drug biotransformation enzyme.

22. The human cell model of claim 9, wherein the cells comprise variable amounts of said expression vectors comprising ectopic DNA sequences coding for the drug biotransformation enzymes selected from among Phase I drug biotransformation enzymes and Phase II drug biotransformation enzymes.

23. A kit comprised of one or more expression vectors coding for the sense and anti-sense mRNA of the Phase I and Phase II drug biotransformation enzymes.


SEQUENCE LISTING (22 sequences)

SEQ ID NO: 1 - DNA, 23 bp, Artificial Sequence
  Synthetic Construct
cctccaggatccctacactgatc

SEQ ID NO: 2 - DNA, 22 bp, Artificial Sequence
  Synthetic Construct
cccggatcccagatagcaaaac

SEQ ID NO: 3 - DNA, 27 bp, Artificial Sequence
  Synthetic Construct
gcaggtaccgttggtaaagatggcatt

SEQ ID NO: 4 - DNA, 27 bp, Artificial Sequence
  Synthetic Construct
agccatggaccggagtcttaccaccac

SEQ ID NO: 5 - DNA, 26 bp, Artificial Sequence
  Synthetic Construct
cccgaattcaccatgctggcctcagg

SEQ ID NO: 6 - DNA, 26 bp, Artificial Sequence
  Synthetic Construct
ccgaattccagacctgcaccggcaca

SEQ ID NO: 7 - DNA, 27 bp, Artificial Sequence
  Synthetic Construct
cagggatcccagaccaggaccatggaa

SEQ ID NO: 8 - DNA, 28 bp, Artificial Sequence
  Synthetic Construct
tttgggatccttccctcagccccttcag

SEQ ID NO: 9 - DNA, 28 bp, Artificial Sequence
  Synthetic Construct
ggggtaccttcaatggaaccttttgtgg

SEQ ID NO: 10 - DNA, 26 bp, Artificial Sequence
  Synthetic Construct
cccaagcttgcattcttcagacaggg

SEQ ID NO: 11 - DNA, 30 bp, Artificial Sequence
  Synthetic Construct
ggaattcggcttcaatggattctcttgtgg

SEQ ID NO: 12 - DNA, 28 bp, Artificial Sequence
  Synthetic Construct
cgtctagacttcttcagacaggaatgaa

SEQ ID NO: 13 - DNA, 26 bp, Artificial Sequence
  Synthetic Construct
cccgaattcaccatgctggcctcagg

SEQ ID NO: 14 - DNA, 26 bp, Artificial Sequence
  Synthetic Construct
ccgaattccagacctgcaccggcaca

SEQ ID NO: 15 - DNA, 21 bp, Artificial Sequence
  Synthetic Construct
atggatccttttgtggtcctt

SEQ ID NO: 16 - DNA, 19 bp, Artificial Sequence
  Synthetic Construct
agcagccagaccatctgtg

SEQ ID NO: 17 - DNA, 22 bp, Artificial Sequence
  Synthetic Construct
ctaagggaacgacactcatcac

SEQ ID NO: 18 - DNA, 22 bp, Artificial Sequence
  Synthetic Construct
ctcaccaggaaagcaaagacac

SEQ ID NO: 19 - DNA, 26 bp, Artificial Sequence
  Synthetic Construct
gttgaagaatccaagtggcgatggac

SEQ ID NO: 20 - DNA, 27 bp, Artificial Sequence
  Synthetic Construct
acagaatccttgaagaccaaagtagaa

SEQ ID NO: 21 - DNA, 27 bp, Artificial Sequence
  Synthetic Construct
ccaggatcctgctatcatggcagagaa

SEQ ID NO: 22 - DNA, 33 bp, Artificial Sequence
  Synthetic Construct
tatggatcccaaaactttagaacattggtattg